(19) United States
(12) Patent Application Publication
(10) Pub. No.: US 2015/0374402 A1
(43) Pub. Date: Dec. 31, 2015

(54) SURGICAL BLADE

(71) Applicant: University of Utah Research Foundation, Salt Lake City, UT (US)

(72) Inventor: Randall J. Olson, Salt Lake City, UT (US)

(21) Appl. No.: 14/749,159

(22) Filed: Jun. 24, 2015

Related U.S. Application Data
(60) Provisional application No. 62/017,587, filed on Jun. 26, 2014.

(51) Int. Cl. A61B 17/3211; A61F 9/013

(52) U.S. Cl. CPC A61B 17/3211; A61F 9/0133; A61B 2017/00982

(57) ABSTRACT

A surgical cutting device may be made of one or more pieces and include a front edge having a plurality of incision points. The incision points may be the forwardmost points of the front edge and may be separated by a recessed portion. The surgical cutting device may also have lateral edges that are non-cutting surfaces.

### CROSS-REFERENCE TO RELATED APPLICATIONS

This application claims priority to and the benefit of U.S. Provisional Application Ser. No. 62/017,587, entitled “SURGICAL BLADE”, filed on Jun. 26, 2014, which application is incorporated by reference in its entirety herein.

### BACKGROUND OF THE INVENTION

1. The Field of the Invention

Generally, this disclosure relates to surgical tools. More specifically, the present disclosure relates to surgical cutting devices and even more specifically to unidirectional surgical cutting devices.

2. Background and Relevant Art

Medical professionals use specialized tools for particular procedures. For example, shears, scalpels, saws, and diamond blades are all cutting devices used in surgery, but each has a specific purpose. Scalpels alone are made in hundreds of configurations and sizes for different applications. An appropriate cutting device may increase the likelihood of a procedure resulting in a positive outcome.

For procedures performed on small structures or on complex systems, a small, sharp blade may provide a medical professional with greater levels of control for precision cuts and movements. For example, diamond blade cutting devices may be entirely or primary synthetic diamond. The synthetic diamond may have a very high hardness. The high hardness may allow the synthetic diamond to be sharpened to a fine point and allow the fine point to resist deformation and wear better than other materials.

A diamond blade may be so sharp that it may cut through material which the medical professional does not intend to cut. The cutting direction may not be in the apparent direction of movement. A blade may be sharp enough that the cut may inadvertently occur, for example, perpendicular to the direction of motion if the patient moves or the hand shakes.

Diamond blades are used, for example, in ocular surgery. The diamond blade is used to make incisions through the sclera and cornea. An incision is made by advancing the diamond blade in a forward direction to create a longitudinal incision. The incision is made at an angle and a plurality of turns is made during the advancement of the blade by moving the blade normal to the surface of the eye. The sclera is prone to “tear outs” during the advancement and turning of the blade.

Unintentional cuts during ocular surgery lead to a variety of complications ranging from increased recovery times after surgery to blindness. Incorrect cuts can lead to a leaky incision, which results in a 44 fold increase in the risk of endophthalmitis. Incorrect cuts can also lead to conjunctival ballooning. Conversely, a correct cut will “self-seal” due to intraocular pressure sealing the incision without the use of sutures, which further reduces the likelihood of complications.

### BRIEF SUMMARY OF THE INVENTION

Implementations of the present disclosure relate to apparatuses, systems, and methods for making incisions during surgical procedures.

In a non-limiting embodiment, a device for making incisions includes a body having a front end and a first lateral side and a second lateral side. The front end includes a cutting edge that has a plurality of incision points. The first and second lateral sides have non-cutting edges.

In another non-limiting embodiment, a surgical cutting device includes a body having a front edge, a first lateral edge, and a second lateral edge. The second lateral edge is substantially parallel to the first lateral edge. The first lateral edge includes a non-cutting surface. A plurality of incision points are located at the front edge with at least one of the plurality of incision points adjacent the first lateral edge. A midpoint is located on the front edge between two of the plurality of incision points. The midpoint is recessed toward a center of the body. The device also includes an incision marking to denote an incision length.

Additional features and advantages of exemplary implementations of the disclosure will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims, or may be learned by the practice of such exemplary implementations as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. For better understanding, the like elements have been designated by like reference numbers throughout the various accompanying figures. While some of the drawings are schematic representations, at least some of the figures may be drawn to scale. Understanding that these drawings depict only typical embodiments of the invention and are not therefore to be considered to be limiting of its scope, the invention will be described and explained with additional specificity and detail through the use of the accompanying drawings in which:

FIG. 1 illustrates an embodiment of a surgical cutting device according to the present disclosure;

FIG. 2 illustrates an embodiment of a surgical cutting device having a body including a plurality of beams;

FIG. 3 illustrates an embodiment of a surgical cutting device having an asymmetrical front edge;

FIG. 4 illustrates an embodiment of a surgical cutting device having a plurality of non-contacting cutting surfaces;

FIG. 5 illustrates an embodiment of a surgical cutting device including undercut portions of the body;

FIG. 6 illustrates an embodiment of a surgical cutting device including cutting surfaces adjacent a plurality of incision points;

FIG. 7 illustrates an embodiment of a surgical cutting device having a lateral cutting surface in the front edge;

FIG. 8 illustrates an embodiment of a surgical cutting device having a curved front edge;

FIG. 9 illustrates an embodiment of a surgical cutting device having a front edge with three cutting surfaces;

FIG. 10 illustrates a cross-sectional side view of an embodiment of a cutting surface according to the present disclosure;

FIG. 11 illustrates a cross-sectional side view of an embodiment of a cutting surface having a plurality of angled faces;

FIG. 12 illustrates an embodiment of a surgical cutting device including one or more incision markings;

FIG. 13 illustrates a top view of a longitudinal incision made into an anterior chamber using a surgical cutting device according to the present disclosure;

FIG. 14 illustrates a side cross-sectional view of a longitudinal incision made into an anterior chamber using a surgical cutting device according to the present disclosure;

FIG. 15 illustrates a side detail view of a longitudinal incision made into an anterior chamber using a surgical cutting device according to the present disclosure;

FIG. 16 illustrates a side detail view of a longitudinal incision providing access to an anterior chamber;

FIG. 17 illustrates a side view of a “belt loop” incision made in a sclera using a surgical cutting device according to the present disclosure;

FIG. 18 illustrates a belt inserted through a plurality of belt loop incisions made in a sclera using a surgical cutting device according to the present disclosure; and

FIG. 19 illustrates an embodiment of a surgical cutting device having a front edge with a perpendicular portion.

### DETAILED DESCRIPTION

One or more implementations of the present invention relate to surgical devices. For specifically, one or more implementations of the present invention relate to surgical cutting devices.

A surgical cutting device may have one or more cutting surfaces. Each cutting surface may be used by a medical profession to make an incision normal to the cutting edge or at an angle to the cutting surface. The cutting surface may then cleave a target area as the cutting surface is moved normal to the target area. The cutting surface may also be drawn laterally across the target area while a force is applied normal to the target area. Drawing the cutting edge across a target area may reduce the force necessarily to cleave a material, but may also result in an incision that is larger than necessary for the surgical procedure.

A surgical cutting device may have a blade including a front edge and first and second lateral edges. The front edge may have one or more cutting surfaces. The first and second lateral edges may be non-cutting surfaces. The one or more cutting leading surfaces may cleave the desired material to produce an incision. The non-cutting lateral surfaces may provide lateral stability as the leading cutting surface penetrates a target area, but the non-cutting lateral surfaces may not expand the incision during the procedure. The one or more leading cutting surfaces may be angled or curved such that a front edge of the surgical cutting device has a recessed portion between a first and second point at either end of the front edge.

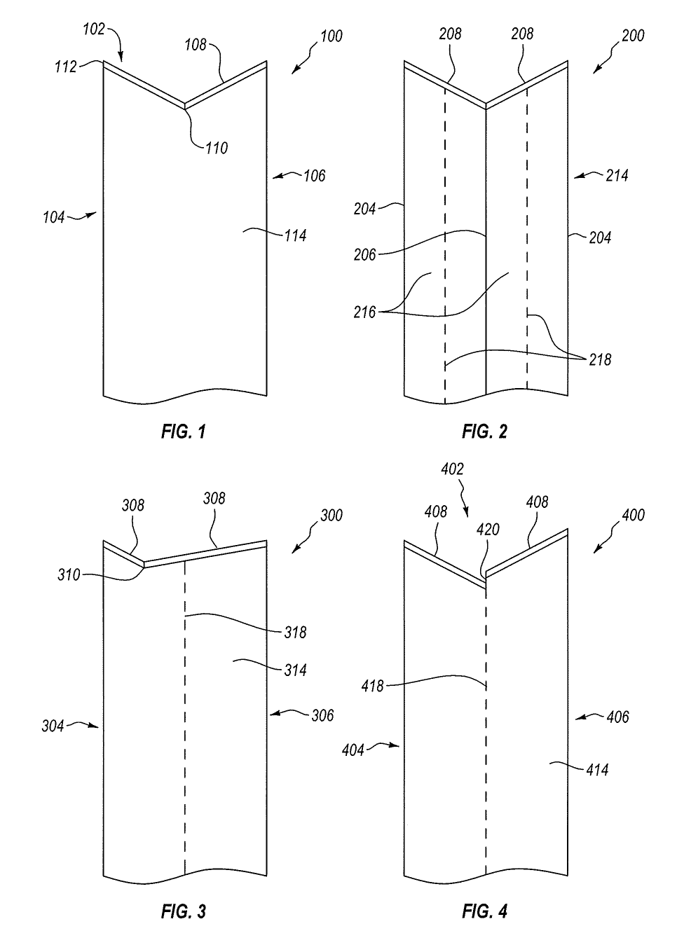

FIG. 1 illustrates an embodiment of a surgical cutting device 100 according to the present disclosure. The surgical cutting device 100 may have a front edge 102, a first lateral edge 104, and a second lateral edge 106. The front edge 102 may include a plurality of cutting surfaces 108. The first lateral edge 104 and second lateral edge 106 may include non-cutting surfaces. FIG. 1 depicts the surgical cutting device 100 including two cutting surfaces 108 that meet at a midpoint 110. The first lateral edge 104 and the second lateral edge 106 may be substantially parallel to one another. The midpoint 110 may be equidistant between the first and second lateral edge 104, 106. The front edge 102 may have a plurality of incision points 112. Each incision point 112 may be located at an end of the front edge 102 and adjacent to the first lateral edge 104 or second lateral edge 106. The incision points 112 may be the portions of the front edge 102 that make contact with a target area to be cut before other portions of the front edge 102. The body 114 of the surgical cutting device 100 may be a single, integral piece.

The body 114 may include diamond, boron nitride, carbon nitride, boron carbon nitride, other superhard materials (i.e. materials over 40 GPa when measured by the Vickers hardness test), titanium, steel, other metals, other suitable surgical materials, and/or combinations thereof. The body 114 may include crystalline material and/or amorphous material. The body 114 may be a single crystal. The body 114 may be a polycrystalline material. In an embodiment, the body 114 may include polycrystalline diamond. In another embodiment, the body 114 may include polycrystalline boron nitride. In yet another embodiment, the body 114 may include amorphous steel. In another embodiment, the body 114 may be single-crystal silicon.

FIG. 2 depicts an embodiment of a surgical cutting device 200 that includes a multi-part body 214. The multi-part body 214 may include a plurality of beams 216. The beams 216 may each have a cutting surface 208 that forms an angle with a longitudinal axis 218 of each beam 216, respectively. The cutting surfaces 208 may abut one another at a midpoint 210, such that the cutting surfaces 208 form a front edge 202 of the surgical cutting device 200 similar to the front edge 102 of the surgical cutting device 100 depicted in FIG. 1. The beams 216 may each have a first lateral edge 204 and a second lateral edge 206. The first and second lateral edge 204, 206 of each beam may be non-cutting surfaces.

FIG. 3 depicts an embodiment of a surgical cutting device 300 that has a midpoint 310 that is not equidistant from a first and second lateral edge 304, 306. The plurality of cutting surfaces 308 may each form different angles with a longitudinal axis 318 of the body 314. For example, one of the cutting surfaces 308 may form an angle with the longitudinal axis 318 having a range of about 15 to about 45 degrees and another of the cutting surfaces 308 may form an angle with the longitudinal axis 318 having a range of about 45 degrees to about 60 degrees. In another example, one of the cutting surfaces 308 may form an angle with the longitudinal axis 318 about 15 degrees and another of the cutting surfaces 308 may form an angle with the longitudinal axis 318 about 45 degrees. In at least embodiment, the differing angles of the cutting surfaces 308 may allow for easier penetration of the target area to be cut.

FIG. 4 depicts a surgical cutting device 400 with a staggered front edge 402. The front edge 402 may include a plurality of cutting surfaces 408. The cutting surfaces 408 may be laterally adjacent to one another. For example, the cutting surfaces 408 may extend laterally across an entire width of the front edge 402 of the surgical cutting device 400 and from a first lateral edge 404 to a second lateral edge 406. However, the cutting surfaces 408 of the front edge 402 may not meet at a midpoint, such as depicted in FIG. 1. The cutting surfaces 408 of the front edge 402 may be separated by a third lateral edge 420. The third lateral edge 420 may be substantially parallel to a longitudinal axis 418 of a body 414 of the surgical cutting device 400. The third lateral edge 420 may include a cutting surface (not shown) or not.

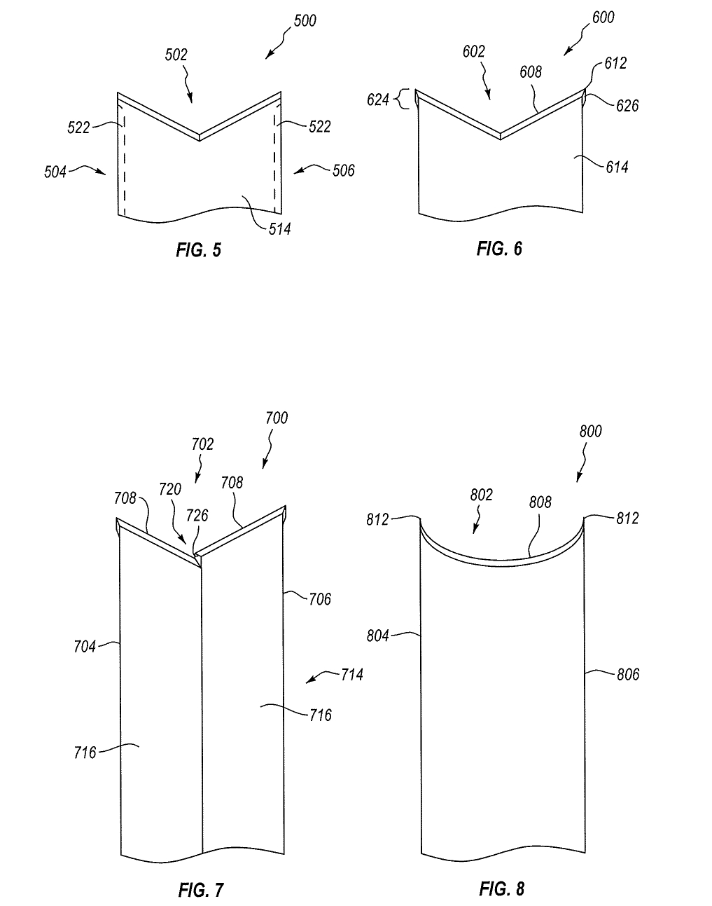

FIG. 5 illustrates the removal of undercut portions 522 from a first lateral edge 504 and a second lateral edge 506 of a surgical cutting device 500. The undercut portion 522 may be removed using a mechanical process, such as abrasive grinding or water jet cutting; a thermal process, such as a cutting torch; an electrical process, such as electrical discharge machining; a chemical process, such as etching; other suitable removal processes depending on the material used for construction of the surgical cutting device 500; and/or combinations thereof. A manufacturing process used to produce the a body 514 may produce first and second lateral edges 504, 506 having portions that are sharp enough to cut a material in the target area. The undercut portion 522 may allow the first and second lateral edges 504, 506 to include a round profile, a flat profile, another non-cutting profile, and/or combinations thereof. The removal of the undercut portion 522 may result in a body 514 having width less than a width of a front edge 502. The undercut portions 522 may be symmetrical about a longitudinal axis 518. The undercut portions 522 may be different on the first lateral edge 504 and the second lateral edge 506. In an embodiment, an undercut portion 522 may have a width less than about 5% of an original width of the body 514. In another embodiment, an undercut portion 522 may have a width less than about 10% of an original width of the body 514. In a particular embodiment, an undercut portion 522 may have a width about 5% of an original width of the body 514.

FIG. 6 depicts a surgical cutting device 600 having overhanging portions 624 of the front edge 602 that extend laterally beyond a width of a body 614. The overhanging portion 624 may be formed by the removal of material from an undercut portion 522 similar to that described in relation to FIG. 5. The overhanging portion 624 may be formed by the addition or accretion of material on the body 614 to widen the front edge 602. For example, material may be added to the body 614 by chemical vapor deposition, metal vapor deposition, physical vapor deposition, ion sputtering, chemical bath deposition, welding, vapor-phase epitaxy, liquid-phase epitaxy, solid-phase epitaxy, molecular-beam epitaxy, other suitable affixation or accretion methods, or combinations thereof. The overhanging portion 624 may include a lateral cutting surface 626 similar to a cutting surface 608 on the front edge 602. In an embodiment, the lateral cutting surface 626 may meet the cutting surface 608 at an incision point 612.

FIG. 7 depicts a surgical cutting device 700 including a body 714 that includes a plurality of beams 716. The beams 716 may each have a cutting surface 708. In an embodiment, the cutting surfaces 708 of the front edge 702 may be separated by a third lateral edge 720. The third lateral edge 720 may be substantially parallel to a first lateral edge 704 and/or a second lateral edge 706 the surgical cutting device 700. The third lateral edge 720 may include a lateral cutting surface 726. In at least one embodiment, the lateral cutting surface 726 on the third lateral edge 720 may allow the surgical cutting device 700 to cut material pressed between the third lateral edge 720 and the cutting surface 708 of the front edge 702.

FIG. 8 depicts a surgical cutting device 800 having a front edge 802 including a curved cutting surface 808. The curved cutting surface 808 may terminate at incision points 812 adjacent a first lateral edge 804 and a second lateral edge 806 at each end of the front edge 802, respectively. In at least one embodiment, the curved cutting surface 808 may allow the incision points 812 to contact a target area to be cut before the remainder of the curved cutting surface 808. The curved cutting surface 808 may cut with little lateral force generated during forward movement of the curved cutting surface 808 through the target area to be cut.

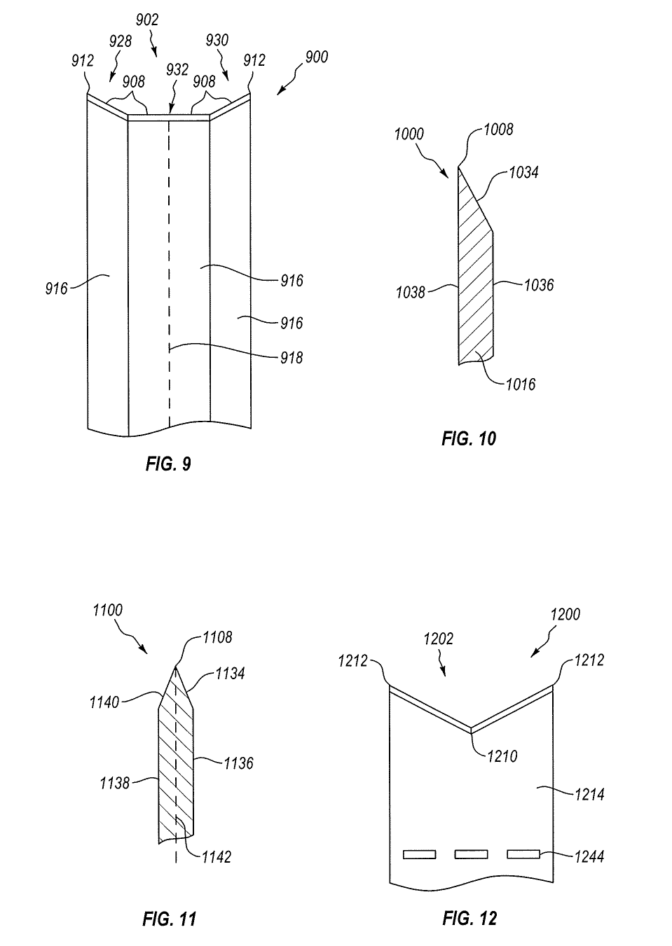

FIG. 9 illustrates another embodiment of a surgical cutting device 900 that includes a plurality of incision points 912 at the two ends of a front edge 902. The front edge 902 may have more than two cutting surfaces. The surgical cutting device 900 depicted in FIG. 9 includes three cutting surfaces with a first angled portion 928 and a second angled portion 930 laterally separated by a center portion 932. The center portion 932 may be substantially perpendicular to a longitudinal axis 918 of the surgical cutting device 900. The center portion 932 may be substantially straight; may be v-shaped, such as depicted in FIG. 1; may be curved, such as depicted in FIG. 8; or may be a combination thereof. Similarly, the first and second angled portion 928, 930 may be substantially straight, v-shaped, curved, or a combination thereof. The center portion 932 and the first and second angled portion 928, 930 may each include a cutting surface 908 thereon. The center portion 932 and the first and second angled portion 928, 930 may be formed in a body made of single contiguous piece. The center portion 932 and the first and second angled portion 928, 930 may each be formed in individual beams 916, respectively, as depicted in FIG. 9.

FIGS. 10 and 11 depict embodiments of cutting surfaces 1008, 1108 of a surgical cutting device 1000, 1100 in cross-section. The profile of a cutting surface 1008 may include an angled face 1034. The angled faced 1034 may extend from a top surface 1036 to a bottom surface 1038 of a body 1016. In an embodiment, the angled face 1034 may form an angle with the bottom surface 1038 less than about 60%. In another embodiment, the angled face 1034 may form an angle with the bottom surface 1038 less than about 45%. In a further embodiment, the angled face 1034 may form an angle with the bottom surface 1038 less than about 30%. In a yet further embodiment, the angled face 1034 may form an angle with the bottom surface 1038 less than about 20%. In an embodiment, the body 1016 may have a thickness less than about 250 micrometers. In another embodiment, the body 1016 may have a thickness less than about 150 micrometers. In a further embodiment, the body 1016 may have a thickness less than about 100 micrometers. In a yet further embodiment, the body 1016 may have a thickness between about 80 micrometers and about 100 micrometers.

FIG. 11 depicts a cutting surface 1108 of a surgical cutting device 1100 having a first angled face 1134 and a second angled face 1140. In an embodiment, the first angled face 1134 may extend from a top surface 1136. The second angled face 1140 may extend from the bottom surface 1138. The surgical cutting device 1100 may have a center plane 1142 that is substantially parallel to the top surface 1136 and the bottom surface 1138. In an embodiment, the first angled face 1134 may form an angle with the center plane 1142 less than about 60%. In another embodiment, the first angled face 1134 may form an angle with the center plane 1142 less than about 45%. In a further embodiment, the first angled face 1134 may form an angle with the center plane 1142 less than about 30%. In a yet further embodiment, the first angled face 1134 may form an angle with the center plane 1142 less than about 20%. In an embodiment, the second angled face 1140 may form an angle with the center plane 1142 less than about 60%. In another embodiment, the second angled face 1140 may form an angle with the center plane 1142 less than about 45%. In a further embodiment, the second angled face 1140 may form an angle with the center plane 1142 less than about 30%. In a yet further embodiment, the second angled face 1140 may form an angle with the center plane 1142 less than about 20%.

The center plane 1142 may be equidistant between the top surface 1136 and the bottom surface 1138. The center plane 1142 may be closer to either the top surface 1136 or the bottom surface 1138. The first angled face 1134 and the second angled face 1140 may intersect one another at a point and/or line within the center plane 1142. The first angled face 1134 and second angled face 1140 may form an angle at or near the cutting surface 1108. In an embodiment, the first angled face 1134 and second angled face 1140 may form an angle less than about 60%. In another embodiment, the first angled face 1134 and second angled face 1140 may form an angle less than about 45%. In a further embodiment, the first angled face 1134 and second angled face 1140 may form an angle less than about 30%. In a yet further embodiment, the first angled face 1134 and second angled face 1140 may form an angle less than about 20%.

In an embodiment, the cutting surfaces 1008, 1108 depicted in FIGS. 10 and 11 may have a sharpness (i.e. radius of curvature of a tip of the cutting surface) less than about 150 nanometers. In still another embodiment the cutting surfaces 1008, 1108 may have a sharpness less than about 100 nanometers or less than about 70 or even about 50 nanometers. In another embodiment, the cutting surfaces 1008, 1108 may have a sharpness less than about 30 nanometers. In a further embodiment, the cutting surfaces 1008, 1108 may have a sharpness less than about 20 nanometers. In a yet further embodiment, the cutting surfaces 1008, 1108 may have a sharpness less than about 10 nanometers.

FIG. 12 depicts a surgical cutting device 1200 according to the present disclosure and including an incision marking 1244 located on a body 1214 of the surgical cutting device 1200. The incision marking 1244 may be made on the body 1214 of the surgical cutting device 1200 by grinding, etching, printing, deposition, other suitable methods of providing indicia, or combinations thereof. The incision marking 1244 may mark a distance from a plurality of incision points 1212. The incision marking 1244 may mark a distance from a midpoint 1210 of the front edge 1202 of the surgical cutting device 1200. In an embodiment, the incision marking 1244 may mark a distance of about 2.0 millimeters. In another embodiment, the incision marking 1244 may mark a distance of about 2.5 millimeters. In a further embodiment, the incision marking 1244 may mark a distance of about 1.5 millimeters. The surgical cutting device 1200 may also include more than one incision marking 1244.

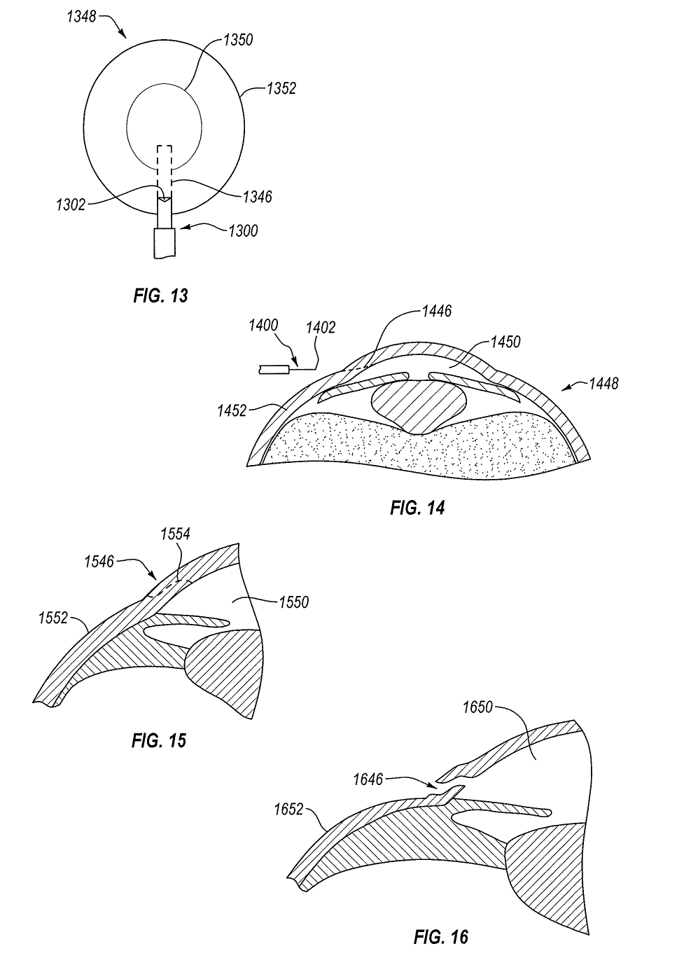

FIGS. 13 and 14 illustrate a surgical cutting device 1300, 1400 according to the present disclosure used to make an incision 1346, 1446 into an eyeball 1348, 1448. The incision 1346, 1446 may be made by urging the surgical cutting device 1300, 1400 forward (i.e. in the direction of the front edge 1302, 1402). A plurality of incision points (for example, the incision points 1212 shown in FIG. 12) of the front edge 1302 may penetrate the target area to be cut first. The remainder of the front edge 1302 may then penetrate the target area to be cut. A midpoint (for example, the midpoint 1210 shown in FIG. 12) may penetrate the target area to be cut.

In an embodiment, the incision 1346, 1446 may extend into the anterior chamber 1350, 1450 of the eyeball 1348, 1448 through the sclera 1352, 1452 and/or cornea. The incision marking 1244, depicted in FIG. 12, may also mark a distance about equal to the desired length of incision 1346, 1446. In an embodiment, the incision 1346, 1446 may be about 2.0 millimeters. In another embodiment, the incision 1346, 1446 may be about 2.5 millimeters. In a further embodiment, the incision 1346, 1446 may be about 1.5 millimeters. In a yet further embodiment, the incision 1346, 1446 may have a length sufficient to penetrate the anterior chamber 1350, 1450.

FIG. 15 illustrates a detail view of an incision 1546 similar to or the same as that shown in FIGS. 13 and 14. The incision 1546 may penetrate through the sclera 1552 and/or cornea in a non-linear fashion before entering the anterior chamber 1550. The incision 1546 may include one or more turns 1554 along the length of the incision 1546. In an embodiment, the one or more incision markings 1244 depicted in FIG. 12 may correspond to the location of the one or more turns 1554 of the incision 1546. The incision 1546 may be made to a first incision marking (similar to incision markings 1244 depicted in FIG. 12), at which point, the angle of the surgical cutting implement relative to the sclera 1552 may be changed and a turn 1554 may be made. The incision 1546 may then proceed to a second incision marking, at which point, the angle of the surgical cutting implement may change again and another turn 1554 may be made. In at least one embodiment, the non-cutting lateral surfaces may allow a medical practitioner to change the angle of the surgical cutting implement more without cutting laterally when compared to a surgical implement having lateral cutting surfaces. The incision 1546 may then proceed to a third incision marking that indicates the incision 1546 is of a predetermined length. FIG. 16 illustrates a completed incision 1646 providing access to the anterior chamber 1650 through the sclera 1652.

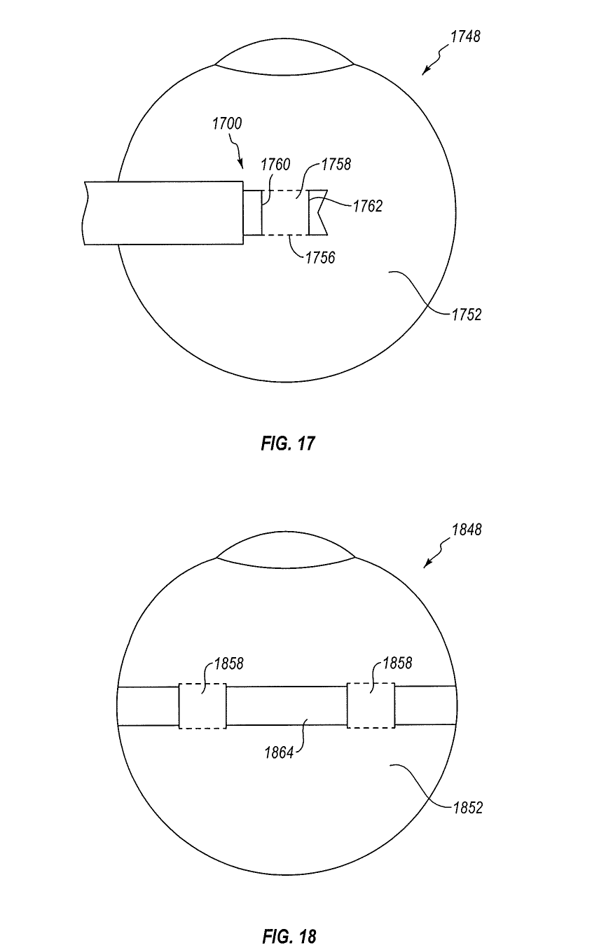

Another example procedure in which a surgical cutting device 1700 according the present disclosure may be used is illustrated in FIGS. 17 and 18. The surgical cutting device 1700 may be urged through the sclera 1752 to form an incision 1756 through the sclera 1752. The incision 1756 may form a “belt loop” 1758. The incision 1756 may have an entrance 1760 into the sclera 1752 and an exit 1762 from the sclera 1752. The incision 1756 may be straight. The incision 1756 may have one or more turns similar to those shown in FIG. 15. The surgical cutting device 1700 may include one or more incision markings (not visible in FIG. 17), similar to the incision markings 1244 described in relation to FIG. 12. The incision markings on the surgical cutting device 1700 may correspond to the locations of the one or more turns in the incision 1756.

As shown in FIG. 18, a medical professional may use a surgical cutting device according to the present disclosure to produce one or more belt loops 1858 in the sclera 1852. The belt loops 1858 may allow the placement of a scleral band 1864 around the eyeball 1848. The scleral band 1864 may provide compression around the eyeball 1848. The belt loops 1858 made using the surgical cutting device according to the present disclosure may support scleral bands 1864 without a need for sutures.

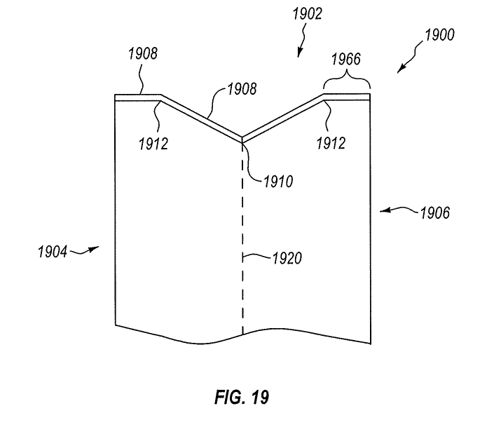

FIG. 19 depicts another embodiment of a surgical cutting device 1900 having a front edge 1902 with perpendicular portions 1966 that may be substantially perpendicular to a longitudinal axis 1920 of the surgical cutting device 1900. The perpendicular portions 1966 may extend from the lateral edges 1904, 1906 inward toward the longitudinal axis 1920 a distance before the front edge 1902 angles backward at the incision points 1912 toward the midpoint 1910. In an embodiment, each perpendicular portion 1966 may be less than or about 100 micrometers. In another embodiment, each perpendicular portion 1966 may be less than or about 150 micrometers. In yet another embodiment, each perpendicular portion 1966 may be less than or about 200 micrometers. In some embodiments, the perpendicular portion 1966 does not include a cutting surface. In other embodiments, the entire front edge 1902 may include cutting surfaces 1908.

The terms “approximately,” “about,” and “substantially” as used herein represent an amount close to the stated amount that still performs a desired function or achieves a desired result. For example, the terms “approximately,” “about,” and “substantially” may refer to an amount that is within less than 10% of within less than 5% of, within less than 1% of, within less than 0.1% of, and within less than 0.01% of a stated amount.

In the description herein, various relational terms are provided to facilitate an understanding of various aspects of some embodiments of the present disclosure. Relational terms such as “bottom,” “below,” “top,” “above,” “back,” “front,” “left,” “right,” “rear,” “forward,” “up,” “down,” “horizontal,” “vertical,” “clockwise,” “counterclockwise,” “upper,” “lower,” and the like, may be used to describe various components, including their operation and/or illustrated position relative to one or more other components. Relational terms do not indicate a particular orientation for each embodiment within the scope of the description or claims. Accordingly, relational descriptions are intended solely for convenience in facilitating reference to various components, but such relational aspects may be reversed, flipped, rotated, moved in space, placed in a diagonal orientation or position, placed horizontally or vertically, or similarly modified. Certain descriptions or designations of components as “first,” “second,” “third,” and the like may also be used to differentiate between identical components or between components which are similar in use, structure, or operation. Such language is not intended to limit a component to a singular designation. As such, a component referenced in the specification as the “first” component may be the same or different than a component that is referenced in the claims as a “first” component.

While embodiments disclosed herein may be used in ocular surgical procedures, such environments are merely illustrative. Systems, devices, assemblies, methods, and other components of the present disclosure, or which would be appreciated in view of the disclosure herein, may be used in other applications and environments. In other embodiments, surgical cutting devices according to the present disclosure may be used in dermatological procedures, vascular procedures, or other suitable surgical procedures.

Furthermore, while the description or claims may refer to “an additional” or “other” element, feature, aspect, component, or the like, it does not preclude there being a single element, or more than one, of the additional element. Where the claims or description refer to “a” or “an” element, such reference is not be construed that there is just one of that element, but is instead to be inclusive of other components and understood as “at least one” of the element. It is to be understood that where the specification states that a component, feature, structure, function, or characteristic “may,” “might,” “can,” or “could” be included, that particular component, feature, structure, or characteristic is provided in some embodiments, but is optional for other embodiments of the present disclosure. The terms “couple,” “coupled,” “connect,” “connection,” “connected,” “in connection with,” and “connecting” refer to “in direct connection with,” or “in connection with via one or more intermediate elements or members.” Components that are “integral” or “integrally” formed include components made from the same piece of material, or sets of materials, such as by being commonly molded or cast from the same material, or commonly machined from the same piece of material stock. Components that are “integral” should also be understood to be “coupled” together.

Although various example embodiments have been described in detail herein, those skilled in the art will readily appreciate in view of the present disclosure that many modifications are possible in the example embodiments without materially departing from the present disclosure. Accordingly, any such modifications are intended to be included in the scope of this disclosure. Likewise, while the disclosure herein contains many specifics, these specifics should not be construed as limiting the scope of the disclosure or of any of the appended claims, but merely as providing information pertinent to one or more specific embodiments that may fall within the scope of the disclosure and the appended claims. Any described features or elements from the various embodiments disclosed may be employed in combination with any other features or elements disclosed herein.

What is claimed is:

1. A surgical cutting device, the device comprising:
a body having a first end and a second end;
a front edge located at the first end, the front edge having a plurality of incision points, the front edge including one or more cutting surfaces;
a first lateral non-cutting edge; and
a second lateral non-cutting edge substantially parallel to the first lateral non-cutting edge.

2. The device of claim 1, further comprising an incision marking.

3. The device of claim 1, further comprising a midpoint located on the front edge, the midpoint being closer to the second end than the incision points.

4. The device of claim 1, wherein at least one of the one or more cutting surfaces is a straight cutting surface.

5. The device of claim 1, wherein at least one of the one or more cutting surfaces is a curved cutting surface.

6. The device of claim 1, wherein at least one of the one or more cutting surfaces is a V-shaped cutting surface.

7. The device of claim 1, wherein the body includes a superhard material.

8. The device of claim 7, wherein the superhard material is diamond.

9. The device of claim 7, wherein the superhard material is a polycrystalline diamond.

10. The device of claim 7, wherein the superhard material is a single-crystal diamond.

11. A surgical cutting device, the device comprising:
a body including
a front edge,
a first lateral edge, wherein the first lateral edge includes a non-cutting surface;
a second lateral edge substantially parallel to the first lateral edge;
a plurality of incision points at the front edge, at least one of the plurality of incision points adjacent the first lateral edge;
a midpoint located on the front edge between two of the plurality of incision points, the midpoint being recessed toward a center of the body; and
an incision marking.

12. The device of claim 11, wherein the midpoint is equidistant from the first lateral edge and the second lateral edge.

13. The device of claim 11, wherein the body include a plurality of beams.

14. The device of claim 11, wherein the front edge comprises a plurality of cutting surfaces.

15. The device of claim 14, wherein at least two of the plurality of cutting surfaces contact one another.

16. The device of claim 11, further comprising a third lateral edge, the third lateral edge located between the first lateral edge and second lateral edge.

17. The device of claim 16, wherein the third lateral edge includes a cutting surface.

18. The device of claim 11, wherein the front edge includes an overhanging portion, the overhanging portion extending beyond the first lateral edge.

19. The device of claim 11, wherein the plurality of incision points are located at a first end and a second end of the front edge.

20. The device of claim 11, further comprising a second incision marking.
